(19) United States
(12) Patent Application Publication
(10) Pub. No.: US 2003/0170651 A1
(43) Pub. Date: Sep. 11, 2003

(54) Method of identifying a polymorphism in CYP2D6

(76) Inventors: Marco Guida, San Diego, CA (US); Linda Benson, Cary, NC (US); Penelope Hopkins, Lakeside, CA (US)

Correspondence Address: SHERIDAN ROSS PC, 1560 BROADWAY, SUITE 1200, DENVER, CO, 80202

(21) Appl. No.: 10/163,598

(22) Filed: Jun. 5, 2002

Related U.S. Application Data
(60) Provisional application No. 60/296,252, filed on Jun. 5, 2001.

(51) Int. Cl. C12Q 1/68; C12P 19/34

(52) U.S. Cl. 435/6; 435/91.2

(57) ABSTRACT

The present invention relates to methods of detecting a polymorphism in a gene encoding xenobiotic metabolizing enzyme CYP2D6. The methodology disclosed is robust and can detect the presence of the wildtype and polymorphic sequences in the presence of gene duplication and gene conversion events.

### REFERENCE TO RELATED APPLICATIONS

This application claims priority under 35 U.S.C. § 119(e) from U.S. Provisional Application Serial No. 60/296,252 filed Jun. 5, 2001, the complete disclosure of which is incorporated herein by reference in its entirety.

### FIELD OF THE INVENTION

The invention resides in the field of accurate detection of polymorphisms in a cytochrome P-450 metabolic enzyme in the presence of changes in genomic organization.

### BACKGROUND OF THE INVENTION

The cytochrome P-450 CYP2D6 enzyme catalyzes the oxidation of a large number of drugs including tricyclic antidepressants, antiarrhythmics, neuroleptics and morphine derivatives. The CYP2D6 gene cluster on chromosome 22 includes two to three related nonfunctional pseudogenes, CYP2D8P, CYP2D 7AP, and CYP2D 7BP, followed by the active gene, CYP2D6. Sixty-eight CYP2D6 polymorphic alleles have been recognized by the P450 Nomenclature Committee, with over thirty alleles associated with alterations in the in vitro or in vivo metabolism of the probe drugs debrisoquine, sparteine, or dextromethorphan. Genetic-based alterations that effect the activity of the CYP2D6 enzyme give rise to the ultrarapid (UM), extensive (EM), intermediate (IM) and poor metabolizer (PM) phenotypes. Individuals homozygous or heterozygous for nonfunctional or partially defective CYP2D6 alleles metabolize these drugs at lower rates, while individuals with duplication of the wildtype allele (CYP2D6*1), other functional alleles such as CYP2D6*35 or the slightly impaired CYP2D6*2 allele metabolize drugs at an increased rate. There are examples at the CYP2D6 locus in which the gene has been duplicated up to as many as 13 copies.

In addition to interindividual variability in CYP2D6 enzyme activity, the incidence of polymorphic metabolism varies among different populations. In particular, differences between Caucasians and Asians are explained by an unequal distribution of CYP2D6 alleles. The defective alleles CYP2D6*3 and CYP2D6*4, that give rise to 85% of the PM phenotype observed in 7% of Caucasians, are found in less than 1% of the Chinese population, explaining the low frequency of PMs in this population. In addition, these two races differ in mean debrisoquine hydoxylase activity within the normal range of CYP2D6 metabolic ratios (MR). In Chinese populations, the mean MR distribution is shifted toward higher values, indicating that an intermediate metabolizer (IM) phenotype predominates. This IM phenotype is associated with the partially defective alleles CYP2D6*10A and CYP2D6*10B. These are the most common alleles (61.5% allele frequency) found in the Chinese population and contain a C to T transition at position 188 that causes a proline to serine amino acid substitution at codon 34 of the CYP2D6 enzyme, leading to the formation of an unstable enzyme which results in lower metabolic activity. This C188T mutation has been associated with a 3-4 fold decreased risk of lung cancer amongst non-smokers in a Chinese population and with alterations in the pharmacokinetics of venlafaxine in a Japanese population.

CYP2D6*10 is a haplotype consisting of four single nucleotide polymorphisms (SNPs) interspersed along the CYP2D6 locus (C188T in exon 1, C1127T in exon 2, G1749C in exon 3, and G4268C in exon 9). Although detection of this haplotype provides important information about individual response to drug therapy and xenobiotics, the genotyping analysis may be complicated by alterations in the genomic organization of the locus which can lead to false genotype calls. Thus there is a need for an accurate method to determine CYP2D6 genotypes in individuals containing genomic alterations of this locus that are known to occur frequently

### SUMMARY OF THE INVENTION

The invention is directed to a method of determining a cytochrome P-450 2D6 genotype of an individual by obtaining genomic DNA from the individual and subjecting a first portion of the genomic DNA to amplification conditions in the presence of a pair of primers. One of the primers hybridizes to genomic DNA comprising a CYP2D6 exon 1 C188T polymorphism and does not hybridize to a CYP2D6 wild-type sequence at position 188 of exon 1. Therefore, the production of an amplification product from this reaction indicates a CYP2D6*10 genotype.

Another embodiment of the present invention is directed to an allele-specific amplification primer, wherein the primer hybridizes to, and primes amplification of, a fragment of a cytochrome P-450 2D6 gene comprising a CYP2D6 exon 1 C188T polymorphism but does not prime amplification of a cytochrome P-450 2D6 gene comprising the wildtype sequence at position 188.

Another embodiment of the invention is a nucleic acid molecule comprising a fragment of a cytochrome P-450 2D6 gene comprising a CYP2D6 exon 1 C188T polymorphism between 10 and 50 nucleotides in length.

Another embodiment of the invention is directed to an amplification product containing the fragment of the CYP2D6 gene between nucleotide 68 and nucleotide 1212. Another embodiment of the invention is the amplification product produced by obtaining genomic DNA of an individual and subjecting at least a portion of the genomic DNA to amplification conditions in the presence of a pair of primers, wherein one of the primers hybridizes to genomic DNA comprising a CYP2D6 exon 1 C188T polymorphism and does not hybridize to a CYP2D6 wild-type sequence at position 188 of exon 1 to produce an amplification product. Similarly, another embodiment of the invention is directed to the amplification product produced by obtaining genomic DNA of an individual and subjecting at least a portion of the genomic DNA to amplification conditions in the presence of a pair of primers, wherein one of the primers hybridizes to genomic DNA comprising a CYP2D6 wild-type sequence at position 188 of exon 1 and does not hybridize to a CYP2D6 exon 1 C188T polymorphism to produce an amplification product.

Another embodiment of the present invention is directed to a method of prescribing a pharmaceutical composition to an individual by obtaining genomic DNA of the individual and subjecting a first portion of the genomic DNA to amplification conditions in the presence of a pair of primers in which one of the primers hybridizes to genomic DNA comprising a CYP2D6 exon 1 C188T polymorphism and does not hybridize to a CYP2D6 wild-type sequence at position 188 of exon 1. The pharmaceutical composition is then prescribed for the individual based on results of the amplification as they indicated the genotype of the individual tested. These genotyping methods and the ability to modify prescriptions based on the results of the methodology are particularly suited for Asian individuals in whom polymorphisms in the CYP2D6 gene occur frequently.

### BRIEF DESCRIPTION OF THE DRAWINGS

FIG. 1. Comparison of electropherograms showing different peak ratios. A) Peak ratios at position 188 in CYP2D6 exon 1. B) Peak ratios at positions 1127, 1749, and 4268 in exons 2, 3, and 9, respectively.

FIG. 2. Comparison of the normal (wild type) nucleotide sequence with that of a common variant in CYP2D6 intron 1.

FIG. 3. Confirmation of the CYP2D6*10 gene duplication by Pulse Field Gel Electrophoresis analysis. Xbal-digested genomic DNA samples from 2 homozygotes *1/*1 (lanes 1 and 2), 2 heterozygotes *1/*10 (lanes 3 and 4) and a homozygote *10/*10 (lane 5) were hybridized with a nonspecific CYP2D probe. The 29Kb and 44Kb bands contain CYP2D6 and CYP2D7P, the 3.5Kb band contain the pseudogene CYP2D8P.

FIG. 4. Allele Specific Amplifications of a *10/*10, a *1/*1, and a *1/*10 sample. Lanes 1, 3, 5 show the result from the ASA assay using a *1-specific forward primer. Lanes 2, 4, 6 show the result when a *10-specific forward primer is used. The control gene is TPMT.

FIG. 5. Mixing experiment using CYP2D6*10 ASA. Lanes 1 and 10: molecular weight markers. Lane 2: equal amount of *1/*1 and *10/*10 genomic DNA were used as PCR templates. Lane 3-9: different DNA ratios of *1/*1 and *10/*10 genomic DNA were used. Ratios are indicated on the bottom.

FIG. 6. Possible origin of the CYP2D6*10-associated gene duplication: an unequal crossing over occurred between CYP2D7P and CYP2D6*10 in a homozygote *10/*10 generating the CYP2D7P-CYP2D6*36-CYP2D6*10 locus (bottom left) and a locus with a deletion spanning from CYP2D7P exon 8 or 9 to CYP2D6 exon 7 or 8 (bottom right).

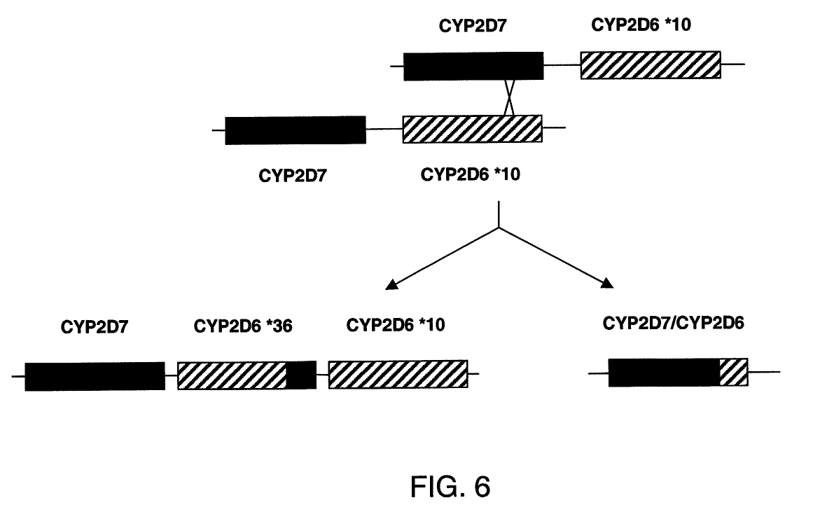

### DETAILED DESCRIPTION OF THE INVENTION

For the sake of clarity, all references in this patent to positions within the CYP2D6 gene will be made with reference to the first nucleotide of the transcription start site as published by Kimura et al. (Am. J. Hum. Genet. 45:889-904, 1989) (Gen Bank Accession No. M33388). Thus, using this numbering system, the codon coding for the start methionine appears at positions 89-91.

An allele consists of a segment of deoxyribonucleic acid (DNA) which comprises all the information needed to become expressed as a polypeptide chain. Thus, alleles differing in nucleotide sequences may give rise to different polypeptide chains or fail to make the protein. However, identical polypeptide chains may be derived from different alleles provided the nucleotide sequence differences are “silent” at the level of translation. Moreover, nucleotide sequence differences between alleles will not affect the polypeptide chain sequences provided the differences occur in introns or in untranslated portions of the exons.

Consequently, alleles recognized as such at the DNA level may not emerge as alleles but as products of the same gene at the protein level. Allelic genes, although similar, differ from each other but occupy identical positions in the genome or at least chromosome. Due to the diploid character of the mammalian genome including the human ones, an individual can only express two alleles at the two given chromosomal loci. However, the entire population may express a large number of alleles at such a locus. Two identical alleles result in a homozygous genotype while two different alleles result in a heterozygous carrier of genetic information.

During sequence analysis of Asian samples which possessed the CYP2D6*10 allele, it was observed that the C and T peaks at position 188 among many heterozygotes were not uniformly equal in height. Investigation of this anomaly using pulse field gel analysis and quantitative cloning revealed that, of 77 Asian samples, the *10 allele occurred with a frequency of 47%. Additionally, 72% of the heterozygote samples with the *10 allele contained multiple copies of the CYP2D6 locus. It was discovered that the amplified CYP2D6*10 allele may contain multiple copies of that allele which can compensate for the decreased CYP2D6 enzymatic activity phenotype in those individuals that are heterozygous or homozygous for the CYP2D6*10 allele. In the presence of these gene duplications, any genotyping assay requiring a pre-amplification of both alleles at the same time will often mask the wild-type sequence at position 188 in the presence of the CYP2D6*10 allele duplication. Thus, genotyping a simple SNP in the CYP2D6 gene may be complicated by alterations in the genomic organization of the locus. This is particularly important with respect to the CYP2D6 genotype as many pharmaceuticals are metabolized by the CYP2D6 enzyme. To better anticipate the efficacy of pharmaceuticals, and to potentially prevent adverse drug reactions based on these individual variations in metabolism, the CYP2D6 genotype of the individual to whom the pharmaceuticals are prescribed may be tested to evaluate the CYP2D6 genotype. The choice of the pharmaceutical prescribed or the dosage of the pharmaceutical prescribed may then be modified based on the CYP2D6 genotype of the individual. This is particularly important in Asian individuals in which the CYP2D6*10 genotype appears frequently but genomic duplication events may have occurred that partially compensate for the CYP2D6*10 phenotype making it difficult to predict the individual response to pharmaceuticals metabolized by the CYP2D6 enzyme. Mutations in the CYP2D6 enzyme have also been linked to an increased susceptibility to cancer. It is suspected that this susceptibility arises following environmental exposure to xenobiotics metabolized by the CYP2D6 enzyme. Indeed, the CYP2D6*10 genotype has been associated with an increased risk of lung cancer. It may be desirable to test certain individuals for CYP2D6 polymorphisms to ascertain the individual susceptibility to cancer based on exposure to certain environmental conditions. Therefore, one embodiment of the present invention is a method of genotyping an individual for a CYP2D6 polymorphism which will correctly identify the CYP2D6 genotype in the presence of an allele-specific CYP2D6 gene duplication.

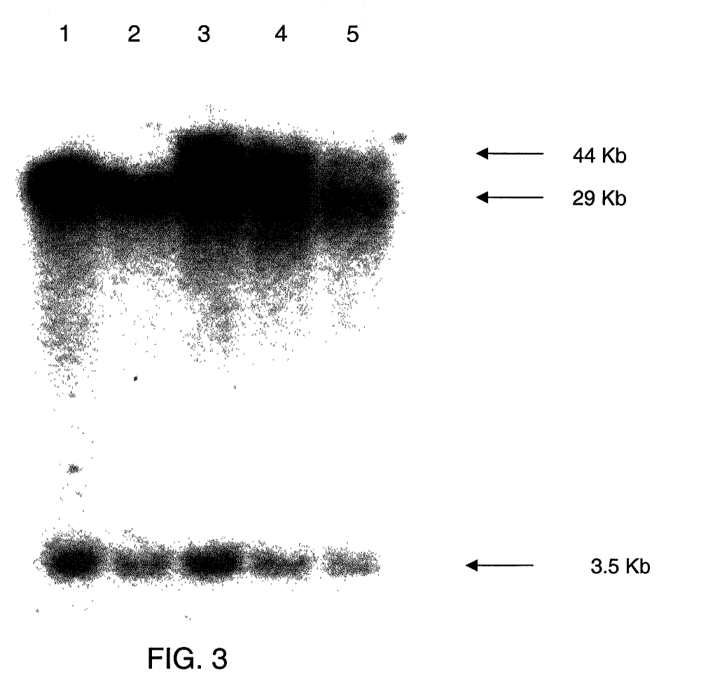

One subvariant of the CYP2D6*10 allele, originally called CYP2D6*10C and later renamed CYP2D6*36, is identical to the CYP2D6*10 allele except for a gene conversion with CYP2D7P in exon 9. The presence of this gene conversion in heterozygote samples was tested by designing a primer pair consisting of a CYP2D6-specific forward primer and a CYP2D7P-specific reverse primer located in exon 9. Tests of heterozygote samples using these PCR primer sets confirmed the presence of the CYP2D6*36 allele. Additionally, sequencing analysis showed unequivocally that approximately 40% of a group of Asian samples tested contain an unrelated polymorphic region in intron 1 (FIG. 3) which may be due to another partial gene conversion to CYP2D7P. This 30 bp-long region includes 7 base pair differences from the CYP2D6 wild-type sequence. Because of these differences, the standard PCR-RFLP primer pair would not amplify any allele that contains the polymorphic region in intron 1. By designing a specific primer pair around this polymorphic region in intron 1, it was possible to develop a method of genotyping an individual for a CYP2D6 polymorphism which will correctly identify the CYP2D6*10 genotype in the presence of a gene conversion between the CYP2D6 and CYP2D7 genes.

One embodiment of the present invention is an allele specific assay (ASA) that detects the wild-type sequence and the CYP2D6*10 allele independently in genomic DNA without the need for an intermediate amplification product. The forward primers are specific for either CYP2D6*1 or CYP2D6*10 while the common reverse primer selects for CYP2D6 and against CYP2D7AP, CYP2D7BP, and CYP2D8P. In a preferred embodiment, the assay includes the amplification of the Thio-purine-methyl-transferase (TPMT) gene to control for assay performance. The assay is a robust assay that can detect the wild-type C188 sequence in the presence of at least twenty-five fold excess T188 copies despite the presence of the exon 9 gene conversion event with the CYP2D7 gene.

The initial step of the allele-specific assay includes amplification of at least a portion of the CYP2D6 gene. Amplification is defined as the production of additional copies of a nucleic acid sequence and is generally carried out using polymerase chain reaction technologies well known in the art [Dieffenbach CW and GS Dveksler (1995) PCR Primer, a Laboratory Manual, Cold Spring Harbor Press, Plainview N.Y.]. As used herein, the term “polymerase chain reaction” (“PCR”) refers to the method of K. B. Mullis U.S. Pat. Nos. 4,683,195 and 4,683,202, hereby incorporated by reference, which describe a method for increasing the concentration of a segment of a target sequence in a mixture of genomic DNA without cloning or purification. The length of the amplified segment of the desired target sequence is determined by the relative positions of two oligonucleotide primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

By allele-specific, it is meant that the assay is capable of detecting the presence or absence of the CYP2D6*10 gene at either CYP2D6 allele independently. The nucleic acids of interest can be amplified from nucleic acid samples using any standard amplification techniques. For instance, polymerase chain reaction (PCR) technology can be used to amplify the sequences of the CYP2D6 genes directly from genomic DNA or from genomic libraries. PCR and other in vitro amplification methods may also be useful, for example, to clone nucleic acid sequences that code for proteins to be expressed, to make nucleic acids to use as probes for detecting the presence of the desired mRNA or DNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990).

The template CYP2D6 gene, or portions thereof, are isolated from the individual to be tested, but need not be purified. The PCR amplification procedure can be performed using purified genomic DNA from an individual, cell lysate, including the genomic DNA of the individual, or other impure sources of genomic DNA. Genomic DNA of the individual subject is isolated by the known methods in the art, such as phenol/chloroform extraction from tissue containing nucleated cells including white blood cells, epithelial cells, etc. The source of the genomic DNA need only be pure enough to allow for amplification of the CYP2D6 gene over the background, nonspecific DNA present in the test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

Two different primer sets are used to amplify the regions of interest on the CYP2D6 gene. In the first set, a forward primer that is specific for CYP2D6*1 is used in combination with a reverse primer that selects for CYP2D6 and against CYP2D7AP, CYP2D7BP, and CYP2D8P. In the second set, a forward primer that is specific for CYP2D6*10 is used in combination with a reverse primer that selects for CYP2D6 and against CYP2D7AP, CYP2D 7BP, and CYP2D8P. Preferably, the reverse primer that selects for CYP2D6 and against CYP2D7AP, CYP2D7BP, and CYP2D8P is the same primer in both amplification reactions. Any means of forming an allele-specific primer known in the art are acceptable for use in the assay. For example, allele-specific primers can be formed by designing a primer having the CYP2D6 sequence with the exception of a mismatch at the polymorphic position 188 at the 3′ end of the primer. Another means for forming an allele-specific primer is to include the CYP2D6 mismatch at the penultimate 3′ position. Thus, the primer has the sequence of the CYP2D6 gene with either a C or T nucleotide at the most 3′ position and with a mismatch at the penultimate 3′ position. Yet another means of forming allele-specific primers is to use modified bases throughout the primer, especially at the most 3′ 4-5 bases of the primer such that the primer hybridizes to only one of the two allele sequences possible at that position. Yet another means of forming a primer that is specific for either the CYP2D6*1 and CYP2D6*10 gene is to design the individual primer to include the position of any one of the four CYP2D6*10 SNPs as the 3′ nucleotide of the primer. For example, the C188T polymorphism may be used to design primers that are specific for either the wildtype CYP2D6*1 or CYP2D6*10 alleles. This is done by designing either forward or reverse primers such that the primer sequence corresponds to the CYP2D6 sequence immediately adjacent the C188T polymorphism. The 3′ nucleotide of the primer is designed to correspond to position 188 of the CYP2D6 gene. The 3′ nucleotide is either a C or a T nucleotide such that if the 3′ position of the primer does not hybridize to the CYP2D6 gene (i.e. if a mismatch occurs at the 3′ position of the primer), the primer will not be extended into a CYP2D6 amplification product. Using this example, the forward CYP2D6*1-specific primer would include the CYP2D6 sequence immediately adjacent to position 188 ( . . . 5′GCACGCTAC3′) terminating 3′ with a C nucleotide corresponding to the wildtype sequence at this position. Conversely, the forward CYP2D6*10-specific primer would include the CYP2D6 sequence immediately adjacent to position 188 ( . . . 5′GCACGCTAC3′) terminating 3′ with a T nucleotide corresponding to the presence of the polymorphism at this position. Similarly, a reverse CYP2D6*1-specific primer would include the CYP2D6 sequence immediately adjacent to position 188 ( . . . 5′GGCCTGGTG3′) terminating 3′ with a G nucleotide corresponding to the wildtype sequence at this position, and a CYP2D6*10-specific primer include the CYP2D6 sequence immediately adjacent to position 188 ( . . . 5′GGCCTGGTG3′) terminating 3′ with an A nucleotide corresponding to the presence of the polymorphism at this position. Any means of forming an allele-specific primer is suitable for the assay methods of the present invention and such primers and the methods of determining the CYP2D6 genotype of an individual using such primers are encompassed here.

The CYP2D6*1- and CYP2D6*10-specific primers maybe used to detect the presence of a CYP2D6*1 or CYP2D6*10 gene respectively. Thus, one embodiment of the present invention is a method of determining the CYP2D6*10 genotype of an individual by the hybridization of allele specific primers to detect the presence of the CYP2D6*10 gene. The primers and/or probes may be of any length sufficient to specifically hybridize to the CYP2D6 gene, ranging from 10 to 500 nucleotides including the length of every integer between 10 and 500. Preferably, the primers and/or probes are at least 10 nucleotides in length, at least 15 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 55 nucleotides in length or at least 60 nucleotides in length.

In a preferred embodiment of the present invention, the products of the amplification of the CYP2D6 allele primed from each primer set are compared to determine the CYP2D6*10 genotype of the individual. If a CYP2D6 gene product is produced only by the first primer set comprising a CYP2D6*1-specific primer, the genotype of the DNA sample tested is wildtype. With respect to this application, the designation of wildtype is used to define a CYP2D6 locus which is not CYP2D6*10. Thus, a PCR product produced only by the first primer set comprising a CYP2D6*1-specific primer is indicative of a wildtype result in the test of the present method although the locus detected could include mutations other than those defining the CYP2D6*10 haplotype. For example, if a PCR product were to be produced by the CYP2D6*1-specific primer set and no PCR product were to be produced by the CYP2D6*10-specific primer set, the individual tested would be identified as wildtype with respect to the results of the method of the present invention despite the fact that the individual may harbor other CYP2D6 genotypes such as CYP2D6*1, CYP2D6*35, CYP2D6*2, CYP2D6*3 or CYP2D6*4. Thus, wildtype, as used in reporting the results of the present test, only indicates the absence of the CYP2D6*10 genotype in the individual tested. If a CYP2D6 gene product is produced only by the second primer set comprising a CYP2D6*10-specific primer, the genotype of the DNA sample tested is homozygous for the CYP2D6*10 genotype. If a CYP2D6 gene product is produced by both the first primer set comprising a CYP2D6*1-specific primer, and the second primer set comprising a CYP2D6*10-specific primer, the genotype of the DNA sample tested is heterozygous for the CYP2D6*10 genotype.

In separate embodiments of the present invention, the method of amplification of the CYP2D6 gene may be conducted simultaneously in the same reaction or separately in independent reactions. The products of the amplification can then be visualized to determine the CYP2D6 genotype of the individual tested. If the amplification products are generated in the same reaction, the CYP2D6*10-specific product may be preferentially amplified if a CYP2D6*10 duplication has taken place. Thus, the methodology of the present invention may not detect the presence of a wildtype allele if the amplification of each allele is conducted in the same reaction and the CYP2D6*10 gene has been duplicated. Typically, the assay in which the wildtype and CYP2D6*10 amplifications are conducted in the same reaction will correctly identify the CYP2D6*10 genotype of the individual tested if the CYP2D6*10 gene has undergone four or fewer duplications. In instances in which the CYP2D6*10 gene has undergone more than four duplications, the amplification of the alleles in the same reaction will mask the presence of a wildtype allele in the case of a heterozygous individual. Thus, the preferred embodiment of the inventive testing methodology includes conducting the CYP2D6*1 and CYP2D6*10 amplifications in separate reactions to assure correct identification of heterozygous individuals in the event of a CYP2D6*10 gene duplication.

Individual sections of the amplified DNA products can also be assayed for the presence of an individual polymorphism of interest. The assay can include any known method of detecting the presence of a polymorphism within the region of the gene in the amplified product. For example, the presence of one or more polymorphisms could be detected by methods such as restriction fragment length polymorphism analysis, direct sequencing analysis of the region, differential hybridization and single strand conformational polymorphism analysis. For example, an amplified section of exon 1 of the CYP2D6 gene can be further analyzed for the presence of a C188T polymorphism by sequencing of the amplification product or restriction fragment length polymorphism (RFLP) analysis.

An embodiment of the present invention further includes the step of prescribing a pharmaceutical composition based on the results of the genotyping assay. A pharmaceutical composition can be any composition, the metabolism of which is affected by the CYP2D6*10 variant. For example, such pharmaceuticals may include lipophilic blockers, antiarrythmic agents, antidepressants, neuroleptics, risperidone, debrisoquine, and venlafaxine. The CYP2D6*10 phenotype typically results in decreased metabolism of pharmaceuticals metabolized by the CYP2D6 enzyme. Thus, results of the genotyping assay that showing the presence of the CYP2D6*10 allele typically results in prescribing a lower dose of the pharmaceutical of interest or the prescribing of a different pharmaceutical with similar properties that is not affected by the altered CYP2D6 phenotype. A lower dose of the pharmaceutical prescribed is a dose that is lower than the dose that would be conventionally prescribed. Conventional dosages for pharmaceuticals metabolized by the CYP2D6 enzyme are well known. See, for example, the dosing guidelines contained in the Physician's Desk Reference (56th edition (Jan. 15, 2002) published by Medical Economics). This method of prescribing a pharmaceutical composition based on the results of the CYP2D6 genotyping assay is particularly preferred for Asian individuals.

The following Examples are provided to illustrate embodiments of the present invention and are not intended to limit the scope of the invention as set forth in the claims.

### EXAMPLES

### Example 1

### Identification of a CYP2D6*10 Gene Duplication

A. Collection of DNA Samples

Blood specimens from 77 healthy and unrelated volunteers from Singapore were collected after obtaining informed consent. All samples were stripped of personal identifiers to maintain confidentiality. Genomic DNAs were extracted from whole blood using Gentra PureGene kit K-50 (Gentra, Minneapolis, Minn., USA). Concentrations of gDNAs were measured on a CytoFluor II fluorometer (PerSeptive Biosystems, Framingham, Mass., USA) using pico green against a standard curve of known concentrations of human placental DNA.

B. Polymerase Chain Reaction Amplification of Genomic DNA Sequences

All polymerase chain reaction (PCR) amplifications were performed using the Perkin Elmer GeneAmp PCR kit (Perkin Elmer Cetus, Norwalk, Colo., USA) according to manufacturer's instructions in 50 μl reactions with Taq Gold DNA polymerase and 100 ng of genomic DNA as template. Magnesium concentrations for each PCR were optimized empirically. The following primers were used for the PCR-RFLP assay:

| | | |
|---|---|---|
| FWD | 5′CCATTTGGTAGTGAGGCAGGTATG3′, | [SEQ ID NO: 1] |
| REV | 5′CACCATCCATGTTTGCTTCTGGT3′. | [SEQ ID NO: 2] |

For each reaction, the magnesium concentration was 1.5 mM. The PCR products were then digested with HphI and run on a 2% agarose gel. For the Allele Specific Amplification (ASA) assay, Master Mix Buffer E (Epicentre Technologies, Madison, Wis., USA) was used in conjunction with the following primers:

| | |
|---|---|
| FWD(wild-type) | |
| 5′GGGCTGCACGCTACC3′ or | [SEQ ID NO: 3] |
| FWD(*10) | |
| 5′TGGGCTGCACGCTACT3′ | [SEQ ID NO: 4] |
| REV | |
| 5′AGCTCGGACTACGGTCATC3′. | [SEQ ID NO: 5] |

The internal control gene primers were:

| | | |
|---|---|---|
| FWD | 5′CTCATCTCCTGAAAGTCCCTGATA3′ | [SEQ ID NO: 6] |
| REV | 5′CCCAGGTCTCTGTAGTCAAATCC3′. | [SEQ ID NO: 7] |

The PCR templates for sequencing CYP2D6 exons 1, 2, 3, and 9 were obtained by using 1 mM magnesium and the following primers:

exon 1, primer pair A:

| | | |
|---|---|---|
| FWD | 5′AGGTATGGGGCTAGAAGCACTG3′ | [SEQ ID NO: 8] |
| REV | 5′AGGACGTCCCCCAAACC3′ | [SEQ ID NO: 9] |

exon 1, primer pair B:

| | | |
|---|---|---|
| FWD | 5′CCTGCCTGGTCCTCTGTGC3′ | [SEQ ID NO: 10] |
| REV | 5′CGTGGGTCACCAGCGC3′ | [SEQ ID NO: 11] |
| exon 2 | | |
| FWD | 5′ACCCACGGCGAGGACA3′ | [SEQ ID NO: 12] |
| REV | 5′CTAGTGCAGGTGGTTTCTTGGC3′ | [SEQ ID NO: 13] |
| exon 3 | | |
| FWD | 5′CTAATGCCTTCATGGCCAC3′ | [SEQ ID NO: 14] |
| REV | 5′GGAGTGGTTGGCGAAGG3′ | [SEQ ID NO: 15] |
| exon 9 | | |
| FWD | 5′AGCTTCTCGGTGCCCACT3′ | [SEQ ID NO: 16] |
| REV | 5′ACGTACCCCTGTCTCAAATGC3′. | [SEQ ID NO: 17] |

The CYP2D6*36-specific PCR amplification was performed at 1 mM magnesium using the following primers:

| | | |
|---|---|---|
| FWD | 5′GGCAAGAAGGATTGTCAGG3′ | [SEQ ID NO: 18] |
| REV | 5′GGCGTCCACGGAGAAGC3′. | [SEQ ID NO: 19] |

Thermal cycling was performed in a GeneAmp PCR System 9700 PCR machine (Perkin Elmer) with an initial denaturation step at 95° C. for 10 minutes, followed by 35 cycles of denaturation at 95° C. for 30 sec, primer annealing at 60° C. for 45 sec, and primer extension at 72° C. for 2 minutes, followed by final extension at 72° C. for 5 minutes, with the following exceptions: the PCR templates for RFLP were amplified at 65° C. for 30 cycles; 62° C. was used as annealing temperature for exon 3; the ASA PCR was performed at 64° C. for 30 cycles; and 58° C. and 40 cycles were used to amplify the CYP2D6*36-specific product.

C. DNA Sequencing

PCR products were prepared for sequencing by spin column purification using Microcon-100 columns (Millipore, Bedford, Mass., USA). Cycle sequencing was performed on the GeneAmp PCR System 9600 PCR machine (Perkin Elmer) using the ABI Prism dRhodamine Terminator Cycle Sequencing Ready Reaction Kit (Applied Biosystems, Foster City, Calif., USA) according to the manufacturer's directions. The sequencing reactions were subjected to 30 cycles at 96° C. for 20 sec, 50° C. for 20 sec, and 60° C. for 4 minutes, followed by ethanol precipitation. Samples were evaporated to dryness at 50° C. for approximately 15 minutes and resuspended in 2 μl of loading buffer (5:1 deionized formamide:50 mM EDTA pH 8.0), heated to 65° C. for 5 minutes, and electrophoresed through 4% polyacrylamide/6M urea gels in an ABI 377 Nucleic Acid Analyzer according to the manufacturer's instructions for sequence determination.

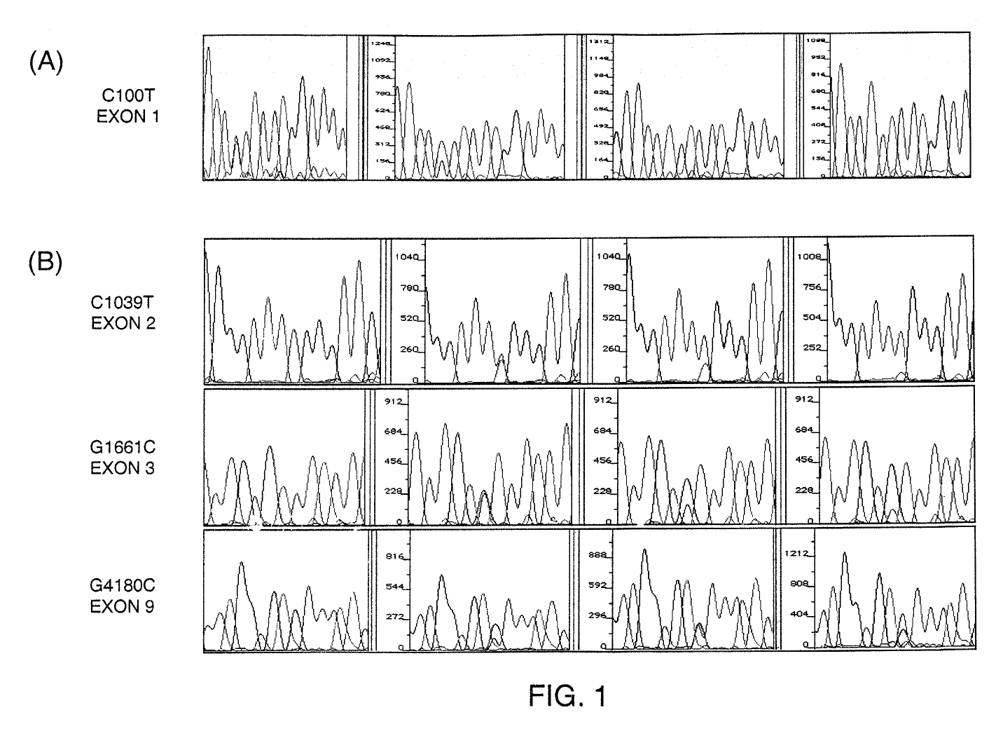

Sequence verification of control samples from a Chinese population with at least one CYP2D6*10 allele revealed that amongst the heterozygotes at position 188 in CYP2D6 exon 1, the peak ratios were not uniformly equal in height. Electropherograms obtained by fluorescence based sequence detection are highly reproducible (±10%) as are heterozygote peak ratios. This analysis clearly shows that the heterozygote samples can be classified based on peak ratios as T=C, T>C, T>>C and T>>>C. (see FIG. 1A). In order to rule out the possibility of a differential allelic amplification due to a polymorphism located in one of the primer binding sites, exon 1 was amplified using two different primer pairs (A and B). The unequal peak ratios at position 188 were observed in both amplifications. Furthermore, since CYP2D6*10 is a haplotype consisting of four SNPs interspersed along the CYP2D6 locus (C188T in exon 1, C1127T in exon 2, G1749C in exon 3, and G4268C in exon 9), exons 2,3, and 9 were sequenced in all samples and, as shown in FIG. 1B, it was discovered that the same lack of uniformity in peak ratios observed at position 188 was also present at positions 1127, 1749, and 4268. However, at position 4268 some inconsistencies were observed, more specifically, some of the heterozygotes showing a T peak greater than the C peak at position 188 were G=C at position 4268. It was calculated that 72% of the heterozygotes have the T peak greater than the C peak.

D. Confirmation of Sequencing Discrepancy

It was confirmed that the unequal peak ratios were not the result of some sequencing artifact by cloning the PCR products from one T>>C (861) and one T=C (870) heterozygote by identifying the number of T clones and the number of C clones generated by each allelic form. Sixty-four colonies were picked per each cloned sample. Fifty-two percent of the colonies from sample 870 were found to have a C at position 188 while only thirty percent of the colonies from sample 861 had a C in the same position. Unequal sequencing peak ratios can also be the result of a polymorphic gene duplication that gives some individuals a greater gene copy number.

E. Cloning

A DNA fragment comprising the region containing the polymorphic site at position 188 in CYP2D6 exon 1 was PCR amplified from 100 ng of genomic DNA isolated from samples 860 and 871, using the primer and PCR conditions previously described. The PCR products were then used directly for subcloning into the TA vector pCR2.1 (Invitrogen, Carlsbad, Calif.) according to manufacturer's instructions. These vectors containing the CYP2D6 inserts could then be used for sequencing the PCR product or the generation of probes.

### Example 2

### Verifying Elevated Gene Copy Number

A. Genomic DNA Digestion

Two wild-type samples (*1/*1), two heterozygotes *1/*10 with T>>C peak ratios, and one *10/*10 homozygote were digested with XbaI. In each case, ten micrograms of genomic DNAs were digested overnight with XbaI at 37° C.

B. Blotting and Hybridization.

The digested samples were electrophoresed on a 1% SeaKem LE agarose gel (FMC BioProducts, Rockland, Me., USA) in a 0.5X TBE buffer using a pulse field gel apparatus (Bio-rad Laboratories, Hercules, Calif.) for 12 hours according to manufacturer's instructions, transferred to Hybond N-Plus membranes (Amersham Pharmacia Biotech, Piscataway, N.J., USA) in 0.4 M NaOH/1 M NaCl transfer buffer, and fixed by UV cross-linking and baking in a vacuum oven. Blots were prehybrydized for 1 hour at 65° C. in 500 mM sodium phosphate buffer containing 7% SDS, 1 mM EDTA, and 10 g/L bovine serum albumin and then hybridized with a gel-purified, radioactively-labeled 500 bp PCR-generated CYP2D6 probe. After labeling, the probe was purified on a G-50 Sephadex spin column (Amersham Pharmacia Biotech) added to the prehybridized blots, and allowed to hybridize overnight at 65° C. Blots were washed once in a 30 mM sodium citrate buffer containing 3 mM NaCl and 0.1% SDS for 15 minutes at 65° C. followed by a wash in a 15 mM sodium citrate buffer containing 1.5 mM NaCl and 0.1% SDS at 65° C. for 15 minutes and a final wash in a 7.5 mM sodium citrate buffer containing 0.75 mM NaCl and 0.1% SDS at 65° C. for 15 minutes. Hybridization bands were revealed by auto-radiography.

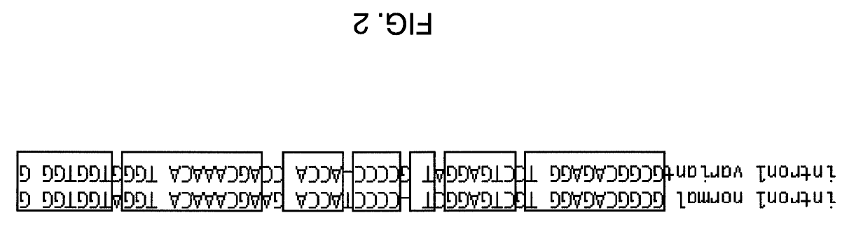

XbaI is known to produce a 29Kb restriction fragment that includes CYP2D6 and the pseudogene CYP2D7P and a 3.5Kb fragment containing the pseudogene CYP2D8P. Given the high homology (>80%) between CYP2D6 and the two pseudogenes and the hybridization conditions used, the probe should have hybridized equally to the three loci. Two hybridization bands (29 and 3.5 Kb) were observed from the homozygotes while the heterozygotes and the *10 homozygote showed an extra ˜44Kb band (FIG. 2) the size of which is consistent with the presence of one or two extra copies of the CYP2D6 gene.

### Example 3

### Structure of the CYP2D6*10-Associated Gene Duplication

Most of the heterozygotes showing one peak greater than the other at positions 188 (exon1), 1127 (exon 2), and 1749 (exon 3) do not show the same uneven peak ratio at position 4268 in exon 9. One possible explanation is that in these cases, the duplication ends between positions 1749 and 4268. Another possibility is that in these heterozygotes, the PCR product for exon 9 is generated by the amplification of only two of the multiple copies of exon 9. This would occur if, in samples containing the gene duplication, a gene conversion event had taken place at one or more copies of the CYP2D6*10 allele between CYP2D6 and CYP2D7P in exon 9. Were this gene conversion event to have occurred, the PCR product encompassing position 4268 in exon 9 would not have been amplified when the CYP2D6-specific primers were used. It is known that one subvariant of the CYP2D6*10 allele, originally called CYP2D6*10C and later renamed CYP2D6*36, is completely identical to the CYP2D6*10 allele except for a gene conversion with CYP2D7P in exon 9. The presence of this gene conversion in these heterozygote samples was tested by designing a primer pair consisting of a CYP2D6 specific forward primer and a CYP2D7P specific reverse primer located in exon 9. Table 1 shows the results obtained from a homozygote *1/*1 (857), a heterozygote with the T peak greater than the C peak (861), and two homozygotes *10/*10 (866 and 873). The homozygote *1/*1 did not amplify with the hybrid primer pair while the other four samples did, confirming the presence of the CYP2D6*36 allele.

| | 2D6 exon 1 | 2D6 exon 9 | 2D6*36 specific |
|---|---|---|---|
| Pts # | seq results | specific primers | primers |
| 857 | CC | Yes | No |
| 861 | T > > C | Yes (T = C) | Yes |
| 866 | TT | Yes | Yes |
| 873 | TT | Yes | Yes |

### Example 4

### Development of a CYP2D6*10 Genotyping Assay

A. Validation of the Standard Assay

In the course of developing and validating a PCR-RFLP assay based on the standard methods published by Wang et al (Molecular basis of genetic variation in debrisoquin hydroxylation in Chinese subjects: polymorphism in RFLP and DNA sequence of CYP2D6. Clin. Pharmacol. Ther. 53:410-18, 1993) and Gao & Zhang (Gao Y, Zhang Q. Polymorphisms of the GSTM1 and CYP2D6 genes associated with susceptibility to lung cancer in Chinese. Mutat. Res. 444:441-49, 1999), each incorporated herein by reference in their entirety, it was discovered that some of the genotypes were incorrectly identified by the PCR-RFLP assay when compared with the sequencing results. More specifically, some heterozygotes were reported as homozygotes (*10/*10) while some of the homozygote wild-type samples failed to amplify altogether. The sequencing validation test, which utilizes a different primer pair than the one used in the PCR-RFLP assay, showed unequivocally that approximately 40% of the Asian samples tested contain a polymorphic region in intron 1 (FIG. 3). This polymorphic region may be due to a partial gene conversion to CYP2D7P. This 30bp-long region includes 7 base pair differences from the CYP2D6 wild-type sequence and those differences were used to design CYP2D6 specific primers for the PCR-RFLP assay. Therefore, the PCR-RFLP primer pair would not amplify any allele that contains the polymorphic region in intron 1. Furthermore, any assay requiring a pre-amplification of both alleles at the same time could mask the wild-type sequence at position 188 in the presence of the CYP2D6*10 allele duplication. It was confirmed that the validated PCR-RFLP performs correctly when the number of duplications is four or less. However, the test may not detect the wild-type sequence when the number of duplications exceeds four.

B. Validation of the Inventive Method

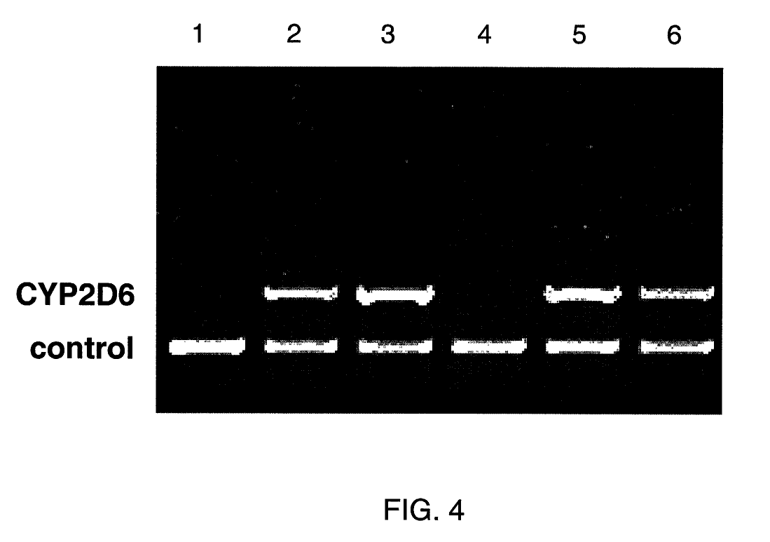

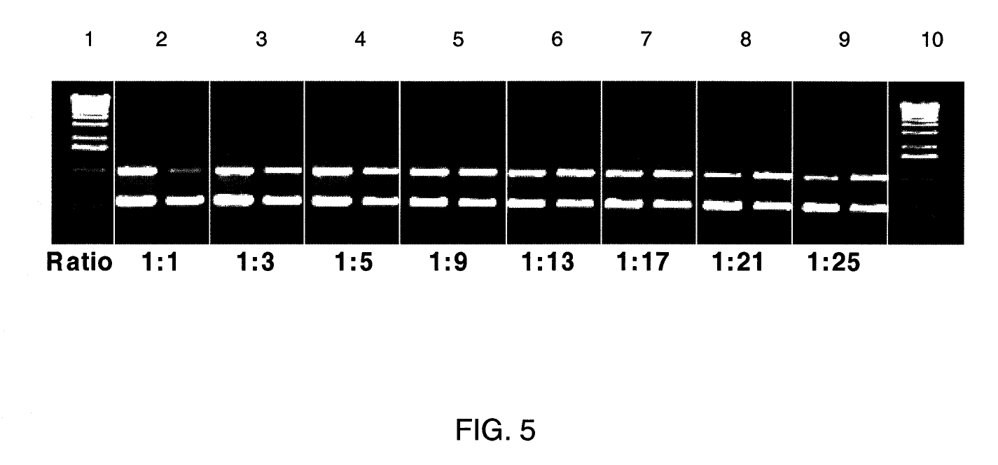

An allele specific assay (ASA) that detects the wild-type sequence and the CYP2D6*10 allele independently in genomic DNA without the need for an intermediate PCR product (FIG. 4) was tested. The forward primers were specific for either CYP2D6*1 or CYP2D6*10 while the common reverse primer selected for CYP2D6 and against CYP2D7AP, CYP2D7BP, and CYP2D8P. The amplification of the Thiopurine methyltransferase (TPMT) gene was also included in the assay to control for assay performance. FIG. 5 shows the result of an experiment in which different ratios of CYP2D6*1 and CYP2D6*10 DNA samples are mixed to simulate varying degrees of duplication. As shown in FIG. 5, even at low ratios of the CYP2D6*1 to CYP2D6*10 alleles, the presence of the wildtype CYP2D6*1 allele was detected by the genotyping assay of the present invention. These results show that the CYP2D6*10 allele specific assay is a robust assay that can detect the wild-type C188 sequence in the presence of at least twenty-five fold excess copies of T188 sequence.

It is to be noted that the term “a” or “an” entity refers to one or more of that entity, including mixtures of the entities of two or more of the entities. As such, the terms “a” (or “an”), “one or more” and “at least one” are used interchangeably herein. It is also to be noted that the terms “comprising,” “including,” and “having” have been used interchangeably.

While various embodiments of the present invention have been described in detail, it is apparent that modifications and adaptations of those embodiments will occur to those skilled in the art. However, it is to be expressly understood that such modifications and adaptations are within the spirit and scope of the present invention, as set forth in the following claims.

What is claimed is:

1. A method of determining a cytochrome P-450 2D6 genotype of an individual comprising:
a) obtaining genomic DNA of the individual, wherein the genomic DNA comprises a CYP2D6 gene;
b) subjecting a first portion of the genomic DNA to amplification conditions in the presence of a pair of primers, wherein one of the primers hybridizes to genomic DNA comprising a CYP2D6 exon 1 C188T polymorphism and does not hybridize to a CYP2D6 wild-type sequence at position 188 of exon 1 and wherein production of an amplification product indicates a CYP2D6*10 genotype.

2. The method, as claimed in claim 1, wherein said primer that hybridizes to genomic DNA comprising a CYP2D6 exon 1 C188T polymorphism and does not hybridize to a CYP2D6 wild-type sequence at position 188 of exon 1 comprises the sequence of SEQ ID NO:4.

3. The method, as claimed in claim 1, wherein said primer that hybridizes to genomic DNA comprising a CYP2D6 exon 1 C188T polymorphism and does not hybridize to a CYP2D6 wild-type sequence at position 188 of exon 1 has the sequence of SEQ ID NO:4.

4. The method, as claimed in claim 1, the other primer has the sequence of SEQ ID NO:5.

5. The method, as claimed in claim 1, wherein the method further comprises:
subjecting a second portion of the genomic DNA to amplification conditions in the presence of a pair of primers, wherein one of the primers hybridizes to genomic DNA comprising a CYP2D6 wild-type sequence at position 188 of exon 1 and does not hybridize to a CYP2D6 exon 1 C188T polymorphism and wherein production of an amplification product indicates a CYP2D6 wild-type genotype.

6. The method, as claimed in claim 5, wherein said primer that hybridizes to genomic DNA comprising a CYP2D6 wild-type sequence at position 188 of exon 1 and does not hybridize to a CYP2D6 exon 1 C188T polymorphism comprises the sequence of SEQ ID NO:3.

7. The method, as claimed in claim 5, wherein said primer that hybridizes to genomic DNA comprising a CYP2D6 wild-type sequence at position 188 of exon 1 and does not hybridize to a CYP2D6 exon 1 C188T polymorphism has the sequence of SEQ ID NO:3.

8. The method, as claimed in claim 5, wherein the other primer has the sequence of SEQ ID NO:5.

9. The method, as claimed in claim 5, wherein production of an amplification product from the first portion of genomic DNA and from the second portion of genomic DNA indicates that said individual is heterozygous for CYP2D6*10 and CYP2D6 wild-type genotypes.

10. The method, as claimed in claim 5, wherein production of an amplification product from the first portion of genomic DNA and the absence of an amplification product from the second portion of genomic DNA indicates that said individual is homozygous for the CYP2D6*10 genotype.

11. The method, as claimed in claim 5, wherein production of an amplification product from the second portion of genomic DNA and the absence of an amplification product from the first portion of genomic DNA indicates that said individual is homozygous for the wildtype CYP2D6 genotype.

12. The method, as claimed in claim 1, wherein said individual is Asian.

13. An allele-specific amplification primer, wherein said primer hybridizes to, and primes amplification of, a fragment of a cytochrome P-450 2D6 gene comprising a CYP2D6 exon 1 C188T polymorphism but does not prime amplification of a cytochrome P-450 2D6 gene comprising the wildtype sequence at position 188.

14. A nucleic acid molecule, wherein said nucleic acid molecule is a fragment of a cytochrome P-450 2D6 gene comprising a CYP2D6 exon 1 C188T polymorphism wherein said nucleic acid molecule is between 10 and 50 nucleotides in length.

15. The nucleic acid molecule, as claimed in claim 14, wherein said CYP2D6 exon 1 C188T polymorphism is at the terminal 3′ position of said nucleic acid molecule.

16. The nucleic acid molecule, as claimed in claim 14, wherein said nucleic acid molecule comprises the sequence of SEQ ID NO:4.

17. The nucleic acid molecule, as claimed in claim 14, wherein said nucleic acid molecule has the sequence of SEQ ID NO:4.

18. An amplification product containing the fragment of the CYP2D6 gene between nucleotide 68 and nucleotide 1212.

19. The amplification product of claim 18, wherein said product contains a C188T mutation.

20. The amplification product produced by the method comprising:
a) obtaining genomic DNA of an individual, wherein the genomic DNA comprises a CYP2D6 gene;
b) subjecting at least a portion of the genomic DNA to amplification conditions in the presence of a pair of primers, wherein one of the primers hybridizes to genomic DNA comprising a CYP2D6 exon 1 C188T polymorphism and does not hybridize to a CYP2D6 wild-type sequence at position 188 of exon 1 to produce an amplification product.

21. The amplification product produced by the method comprising:
a) obtaining genomic DNA of an individual, wherein the genomic DNA comprises a CYP2D6 gene;
b) subjecting at least a portion of the genomic DNA to amplification conditions in the presence of a pair of primers, wherein one of the primers hybridizes to genomic DNA comprising a CYP2D6 wild-type sequence at position 188 of exon 1 and does not hybridize to a CYP2D6 exon 1 C188T polymorphism to produce an amplification product.

22. A method of prescribing a pharmaceutical composition to an individual comprising:
a) obtaining genomic DNA of the individual, wherein the genomic DNA comprises a CYP2D6 gene;
b) subjecting a first portion of the genomic DNA to amplification conditions in the presence of a pair of primers, wherein one of the primers hybridizes to genomic DNA comprising a CYP2D6 exon 1 C188T polymorphism and does not hybridize to a CYP2D6 wild-type sequence at position 188 of exon 1 and wherein production of an amplification product indicates a CYP2D6*10 genotype;
c) subjecting a second portion of the genomic DNA to amplification conditions in the presence of a pair of primers, wherein one of the primers hybridizes to genomic DNA comprising a CYP2D6 wild-type sequence at position 188 of exon 1 and does not hybridize to a CYP2D6 exon 1 C188T polymorphism and wherein production of an amplification product indicates a CYP2D6 wild-type genotype; and,
d) prescribing a pharmaceutical composition for the individual based on amplification results of steps (b) and (c).

23. The method of prescribing a pharmaceutical composition, as claimed in claim 22, wherein said pharmaceutical composition is selected from the group consisting of tricyclic antidepressants, antiarrhythmics, neuroleptics and morphine derivatives.

24. The method of prescribing a pharmaceutical composition, as claimed in claim 22, wherein the step of prescribing a pharmaceutical composition comprises prescribing less than a conventional dose of the pharmaceutical composition metabolized by a CYP2D6 enzyme.

25. The method of prescribing a pharmaceutical composition, as claimed in claim 22, wherein the individual is Asian.


SEQUENCE LISTING (19 sequences)

SEQ ID NO: 1 - DNA, 24 bp, Homo sapiens
ccatttggtagtgaggcaggtatg

SEQ ID NO: 2 - DNA, 23 bp, Homo sapiens
caccatccatgtttgcttctggt

SEQ ID NO: 3 - DNA, 15 bp, Homo sapiens
gggctgcacgctacc

SEQ ID NO: 4 - DNA, 16 bp, Homo sapiens
tgggctgcacgctact

SEQ ID NO: 5 - DNA, 19 bp, Homo sapiens
agctcggactacggtcatc

SEQ ID NO: 6 - DNA, 24 bp, Homo sapiens
ctcatctcctgaaagtccctgata

SEQ ID NO: 7 - DNA, 23 bp, Homo sapiens
cccaggtctctgtagtcaaatcc

SEQ ID NO: 8 - DNA, 22 bp, Homo sapiens
aggtatggggctagaagcactg

SEQ ID NO: 9 - DNA, 17 bp, Homo sapiens
aggacgtcccccaaacc

SEQ ID NO: 10 - DNA, 19 bp, Homo sapiens
cctgcctggtcctctgtgc

SEQ ID NO: 11 - DNA, 16 bp, Homo sapiens
cgtgggtcaccagcgc

SEQ ID NO: 12 - DNA, 16 bp, Homo sapiens
acccacggcgaggaca

SEQ ID NO: 13 - DNA, 22 bp, Homo sapiens
ctagtgcaggtggtttcttggc

SEQ ID NO: 14 - DNA, 19 bp, Homo sapiens
ctaatgccttcatggccac

SEQ ID NO: 15 - DNA, 17 bp, Homo sapiens
ggagtggttggcgaagg

SEQ ID NO: 16 - DNA, 18 bp, Homo sapiens
agcttctcggtgcccact

SEQ ID NO: 17 - DNA, 21 bp, Homo sapiens
acgtacccctgtctcaaatgc

SEQ ID NO: 18 - DNA, 19 bp, Homo sapiens
ggcaagaaggattgtcagg

SEQ ID NO: 19 - DNA, 17 bp, Homo sapiens
ggcgtccacggagaagc